(19) United States
(12) Patent Application Publication
(10) Pub. No.: US 2025/0332072 A1
(43) Pub. Date: Oct. 30, 2025

(54) STRONTIUM-DOPED ROOT CANAL FILLING PASTE, PREPARATION METHOD THEREFOR AND USE THEREOF

(71) Applicant: Guilin Kevin Peter Technology Co., Ltd, Guilin (CN)

(72) Inventors: Xinkuan PANG, Guilin (CN); Changhong WU, Guilin (CN)

(73) Assignee: Guilin Kevin Peter Technology Co., Ltd., Guilin (CN)

(21) Appl. No.: 18/753,690

(22) Filed: Jun. 25, 2024

(30) Foreign Application Priority Data
Apr. 29, 2024 (CN) 2024105318457

(51) Int. Cl. A61K 6/84; A61K 6/54; A61K 6/74; A61K 6/76; A61K 6/853

(52) U.S. Cl. CPC A61K 6/84; A61K 6/54; A61K 6/74; A61K 6/76; A61K 6/853

(57) ABSTRACT

A strontium-doped root canal filling paste, a preparation method therefor and use thereof in the treatment of dental pulp disease are provided. The strontium-doped root canal filling paste includes a component (a) and a component (b), wherein component (a) is selected from calcium silicate compounds, and component (b) is selected from strontium compounds. Component (a) accounts for 20%-40% of by weight of the paste, and component (b) accounts for 0.5%-10% by weight of the paste; and components (a) and (b) are doped in a physical doping manner.

The present application claims the benefit of priority from Chinese patent application CN2024105318457 filed with Chinese Patent Office on Apr. 29, 2024 and entitled “STRONTIUM-DOPED ROOT CANAL FILLING PASTE, PREPARATION METHOD THEREFOR AND USE THEREOF” contents of which are incorporated herein by reference in their entirety.

### TECHNICAL FIELD

The present disclosure relates to the technical field of medical materials, and specifically to a strontium-doped root canal filling paste, a preparation method therefor and use thereof.

### BACKGROUND ART

Mineral trioxide aggregate (MTA), commonly known as root canal cement, is a new calcium silicate-based root canal filling material. In 1998, Mahmoud Torabinejad disclosed a method of repairing a root canal with MTA material for the first time (U.S. Pat. No. 5,769,638). MTA ingredients, similar to those of Ordinary Portland Cement (OPC), mainly include tricalcium silicate (chemical formula Ca₃SiO₅, abbreviated as C₃S), dicalcium silicate (chemical formula Ca₂SiO₄, abbreviated as C₂S), and tricalcium aluminate. The MTA is hydrated when mixed with water and is solidified and hardened through precipitation of colloidal calcium silicate hydrate (C—S—H). The advantages of the MTA as the root canal filling material lie in better biocompatibility and antibacterial property, no shrinkage in curing, and better sealing property. Besides, the MTA is free from the influence of the presence of moisture or blood in curing, has high compressive strength after the curing, and is considered as an optimal root canal filling material at present.

Mixing needs to be performed before use of the conventional MTA, which is not only troublesome but also wasteful. This is improved in the prior art, for example, premixing a calcium silicate compound with an organic solvent to form a paste, while there are many problems with the paste, for example, (1) long setting time and a long treatment period caused by the long setting time; (2) insufficient mineralization property; and (3) poor bonding strength with a root canal wall.

The inventors partially substituted Ca atoms in calcium silicate molecules with Sr atoms to incorporate Sr element into calcium silicate lattices, so as to form a single substance, that is, the inventors chemically doped strontium into the calcium silicate compound to improve the paste (CN113304056B). Although this improvement ameliorates some properties of the paste, comprehensive properties of the paste, such as overall mineralization property, effect of bonding with the root canal wall and setting time, cannot achieve desired effects. In view of this, the present disclosure is specifically proposed.

### SUMMARY

The present disclosure aims at providing a strontium-doped root canal filling paste, a preparation method therefor and use thereof. The strontium-doped root canal filling paste provided in the embodiments of the present disclosure can simultaneously address the problems of defects of the existing strontium-doped root canal filling paste, such as long curing time, low strength, bad mineralization property and insufficient effect of bonding with a root canal wall.

The present disclosure is implemented as follows.

In the first aspect, the present disclosure provides a strontium-doped root canal filling paste which includes a component (a) and a component (b), and wherein the component (a) is selected from calcium silicate compounds, the component (b) is selected from strontium compounds. Component (a) accounts for 20%-40% by weight of the root canal filling paste (hereinafter, the paste), and component (b) accounts for 0.5%-10% by weight of the paste; and component (a) and component (b) are doped in a physical doping manner.

In an optional embodiment, component (a) accounts for 25%-35% by weight of the paste.

Preferably, component (b) accounts for 2-5% by weight of the paste.

In an optional embodiment, the calcium silicate compounds are any one or a combination of any two or more selected from the group consisting of tricalcium silicate, dicalcium silicate and monocalcium silicate.

Preferably, the strontium compound is any one or a combination of any two or more selected from the group consisting of strontium chloride, strontium phosphate, strontium nitrate, strontium fluoride, strontium acetate and strontium ranelate.

In an optional embodiment, the raw materials further include a component (c), which is a radiation inhibitor.

Preferably, the radiation inhibitors are selected from the group consisting of metal oxides, metal sulfate compounds and polyhalogenated C1-C3 alkyls.

Preferably, the radiation inhibitors are selected from the group consisting of transition metal oxides, metal sulfate compounds and polyiodine-substituted C1-C3-alkyls.

Preferably, the radiation inhibitors are any one or a combination of any two or more selected from the group consisting of niobium oxides, zirconium oxides, bismuth oxides, tantalum oxides, barium sulfate and iodoform.

Preferably, component (c) accounts for 30%-40% by weight of the paste, preferably 30%-35%.

In an optional embodiment, the raw materials further include a component (d), which is selected from calcium phosphate compounds, preferably, any one or a combination of any two or more selected from the group consisting of α-tricalcium phosphate, β-tricalcium phosphate, tetracalcium phosphate, calcium dihydrogen phosphate and hydroxyapatite.

Preferably, component (d) accounts for 2%-10% by weight of the paste, preferably 3%-5%.

In an optional embodiment, the paste further includes a component (e), which is selected from organic solvents which can be mixed with component (a) and component (b) to form a paste.

Preferably, the organic solvents are any one or a combination of any two or more selected from the group consisting of alcohol solvents, polymer solvents and sulfoxide solvents.

Preferably, the organic solvents are selected from the group consisting of C2-C5 polyols, poly C2-C5 polyols, polypyrrolidine compounds and C2-C5 sulfoxide solvents.

Preferably, the organic solvents are any one or a combination of any two or more selected from the group consisting of polyethylene glycol, propylene glycol, glycerol, polyvinylpyrrolidone and dimethyl sulfoxide.

Preferably, component (e) accounts for 20%-40% by weight of the paste.

In the second aspect, the present disclosure provides use of the strontium-doped root canal filling paste according to any one of the preceding embodiments in preparation of a material for treatment of dental pulp disease, i.e. pulpitis, and methods of treating dental pulp disease in mammals, preferably humans, in need of such treatment. Within this aspect, the methods of prevention/treatment include applying the strontium-doped root canal filling paste described herein to the root canal region of a mammalian tooth requiring such prevention or treatment.

In the third aspect, the present disclosure provides a preparation method for the strontium-doped root canal filling paste of any one of the preceding embodiments, including: physically mixing the component (a) and the component (b) uniformly.

In an optional embodiment, the preparation method includes: ball-milling and mixing the component (a) and the component (b) to form a mixed powder; and then mixing the mixed powder with at least one of a component (c), a component (d) and a component (e) uniformly.

The present disclosure has the following beneficial effects: for the strontium-doped root canal filling paste provided in the examples of the present disclosure, the calcium silicate compound and the strontium compound are doped in a physical doping manner, which improves mineralization property and biological activity of the strontium-doped root canal filling paste, can greatly shorten setting time of the strontium-doped root canal filling paste, and improve its bondability with the root canal wall, so that the root canal is better sealed.

### BRIEF DESCRIPTION OF DRAWINGS

In order to more clearly illustrate technical solutions of examples of the present disclosure, drawings which need to be used in the examples will be introduced briefly below. It should be understood that the drawings merely show some examples of the present disclosure, and thus should not be considered as limitation to the scope. Those ordinarily skilled in the art still could obtain other relevant drawings according to these drawings, without using any inventive efforts.

FIG. 1 shows a syringe containing a strontium-doped root canal filling paste provided in examples of the present disclosure; and

FIG. 2 shows detection results provided in Detection Example 2 of the present disclosure.

### DETAILED DESCRIPTION OF EMBODIMENTS

In order to make the objectives, technical solutions and advantages of the examples in the present disclosure clearer, the technical solutions in the examples of the present disclosure will be described clearly and completely below. If no specific conditions are specified in the examples, they are carried out under normal conditions or conditions recommended by the manufacturer. If the manufacturers of reagents or apparatus used are not specified, they are conventional products commercially available.

The inventors modified the existing strontium-doped root canal filling paste before by partially substituting Ca atoms in calcium silicate molecules with Sr atoms, to incorporate Sr element into calcium silicate lattices, so as to form a single substance. However, the inventors found that chemically doping strontium into a calcium silicate compound only can improve some properties of the strontium-doped root canal filling paste, but the mineralization property, the effect of bonding with a root canal wall and setting time of the strontium-doped root canal filling paste still cannot be improved at the same time. The inventors further analyzed the strontium-doped root canal filling paste previously improved, for example, explored and studied selection of the doping atoms Sr, a doping method of Sr, a doping amount of Sr, and selection of calcium silicate, and found that doped elements were hard to release after the chemical doping of Sr, and subsequently even if the paste is improved with Sr, the properties of the strontium-doped root canal filling paste still cannot be improved comprehensively. Based on this, the examples of the present disclosure provide a strontium-doped root canal filling paste which includes component (a) and component (b), wherein component (a) is selected from calcium silicate compounds, component (b) is selected from strontium compounds, and components (a) and (b) are doped in a physical doping manner.

The physical doping of the calcium silicate compound and strontium compound can facilitate the Sr element in exerting its efficacy, and can simultaneously improve the formed strontium-doped root canal filling paste in mineralization property, biological activity, setting time, bondability to the root canal wall and so on.

Specifically, component (a) accounts for 20%-40% by weight of the paste. For example, the amount of component (a) can be any numerical value within 20%-40%, such as 20%, 25%, 30%, 35% and 40%, or a range value between any two numerical values, preferably, for example, 25-35%.

Component (b) accounts for 0.5%-10% by weight of the paste. For example, any numerical value within 0.5%-10% can be selected from component (b), such as 0.5%, 1%, 2%, 5%, 7% and 10%, or a range value between any two numerical values, preferably, for example, 2%-5%.

The components (a) and (b) in the above contents can further ensure the properties of the formed strontium-doped root canal filling paste.

Further, the calcium silicate compounds are any one or a combination of any two or more selected from the group consisting of tricalcium silicate, dicalcium silicate and monocalcium silicate; the strontium compound is any one or a combination of any two or more selected from the group consisting of strontium chloride, strontium phosphate, strontium nitrate, strontium fluoride, strontium acetate and strontium ranelate.

The paste can further include a component (c), which is a radiation inhibitor selected from the group consisting of metal oxides, metal sulfate compounds and polyhalogenated C1-C3 alkyls, preferably, transition metal oxides, metal sulfate compounds and polyiodine-substituted C1-C3-alkyls, for example, any one or a combination of any two or more of niobium oxides, zirconium oxides, bismuth oxides, tantalum oxides, barium sulfate and iodoform.

Further, component (c) accounts for 30%-40% by weight of the paste, for example, including any numerical value within 30%-40%, such as 30%, 31%, 32%, 33%, 34%, 35%, 36%, 37%, 38%, 39% and 40%, or a range value between any two numerical values, preferably, for example, 30%-35%.

The paste can further include a component (d) selected from calcium phosphate compounds, for example, including but not limited to any one or a combination of any two or more selected from the group consisting of α-tricalcium phosphate, β-tricalcium phosphate, tetracalcium phosphate, calcium dihydrogen phosphate and hydroxyapatite.

Further, component (d) accounts for 2%-10% by weight of the paste, for example, including any numerical value within 2-10%, such as 2%, 3%, 4%, 5%, 6%, 7%, 8%, 9% and 10%, or a range value between any two numerical values, preferably, for example, 3%-5%.

Further, the paste can further include a component (e) selected from organic solvents which can be mixed with component (a) and component (b) to form a paste. The organic solvents are any one or a combination of any two or more selected from the group consisting of alcohol solvents, polymer solvents and sulfoxide solvents, preferably C2-C5 polyols, poly C2-C5 polyols, polypyrrolidine compounds and C2-C5 sulfoxide solvents. For example, the organic solvents include, but are not limited to, any one or a combination of any two or more of polyethylene glycol, propylene glycol, glycerol, polyvinylpyrrolidone and dimethyl sulfoxide.

Component (e) accounts for 20%-40% by weight of the paste, including for example, any numerical value within 20%-40%, such as 20%, 25%, 30%, 35% and 40%, or a range value between any two numerical values.

In the second aspect, the present disclosure provides use of the strontium-doped root canal filling paste of any one of the preceding embodiments in preparation of a material for treatment of dental pulp disease. This aspect includes methods of treating or preventing dental pulp diseases, i.e. pulpitis, in mammals. The methods include applying the strontium-doped root canal filling paste to the dental pulp/root canal region of a tooth of the mammal in need of such treatment. As will be appreciated by those of ordinary skill, such treatments are typically part of root canal treatments in which infected pulp or suspected infected pulp is removed, the root is cleaned out, and the empty root canal is filled with the strontium-doped root canal filling paste described herein before the affected tooth is sealed.

In the third aspect, the present disclosure provides a preparation method for the strontium-doped root canal filling paste of any one of the preceding embodiments, including: physically mixing component (a) and component (b) uniformly.

Specifically, components (a) and (b) are ball-milled to form a mixed powder, wherein a media-to-material ratio (ball-to-material ratio) of a ball mill is (5-15): 1, a rotational speed is 200 r/min-400 r/min, and then the mixed powder is added, together with at least one of component (c), component (d) and component (e), to a mixer and mixed uniformly, so as to obtain the strontium-doped root canal filling paste. When needed, the strontium-doped root canal filling paste is dispensed into a medical syringe fitted with an injection hose needle, and it is ready for use, for example in the treatment of dental pulp disease and/or root canal therapy.

The characteristics and performances of the present disclosure are further described in detail below in conjunction with examples.

### Example 1-Example 16

Example 1-Example 16 respectively provide a strontium-doped root canal filling paste, of which ingredients are shown in following Table 1 and Table 2.

TABLE 1
Ingredients of Strontium-doped Root Canal Filling Paste of Examples 1-11

| | Example No. | | | | | |
|---|---|---|---|---|---|---|
| Component | Ex. 1 | Ex. 2 | Ex. 3 | Ex. 4 | Ex. 5 | Ex. 6 |
| (a) | 35% | 27% | 35% | 35% | 35% | 35% |
| | tricalcium | tricalcium | dicalcium | tricalcium | tricalcium | tricalcium |
| | silicate | silicate | silicate | silicate | silicate | silicate |
| (b) | 2% strontium | 10% strontium | 2% strontium | 2% strontium | 2% strontium | 2% strontium |
| | phosphate | phosphate | phosphate | chloride | nitrate | fluoride |
| (c) | 33% | 33% | 33% | 33% | 33% | 33% |
| | zirconium | zirconium | zirconium | zirconium | zirconium | zirconium |
| | oxide | oxide | oxide | oxide | oxide | oxide |
| (d) | 5% calcium | 5% calcium | 5% calcium | 5% calcium | 5% calcium | 5% calcium |
| | dihydrogen | dihydrogen | dihydrogen | dihydrogen | dihydrogen | dihydrogen |
| | phosphate | phosphate | phosphate | phosphate | phosphate | phosphate |
| (e) | 25% | 25% | 25% | 25% | 25% | 25% |
| | polyethylene | polyethylene | polyethylene | polyethylene | polyethylene | polyethylene |
| | glycol | glycol | glycol | glycol | glycol | glycol |

| | Example No. | | | | |
|---|---|---|---|---|---|
| Component | Ex 7 | Ex. 8 | Ex. 9 | Ex. 10 | Ex. 11 |
| (a) | 35% | 35% | 35% | 35% | 35% |
| | tricalcium | tricalcium | tricalcium | tricalcium | tricalcium |
| | silicate | silicate | silicate | silicate | silicate |
| (b) | 2% strontium | 2% strontium | 2% strontium | 2% strontium | 2% strontium |
| | ranelate | acetate | phosphate | phosphate | phosphate |
| (c) | 33% | 33% | 33% | 33% | 33% |
| | zirconium | zirconium | zirconium | zirconium | zirconium |
| | oxide | oxide | oxide | oxide | oxide |
| (d) | 5% calcium | 5% calcium | 5% calcium | 5% calcium | 5% calcium |
| | dihydrogen | dihydrogen | dihydrogen | dihydrogen | dihydrogen |
| | phosphate | phosphate | phosphate | phosphate | phosphate |
| (e) | 25% | 25% | 25% | 25% | 25% |
| | polyethylene | polyethylene | polyethylene | glycerol | dimethyl |
| | glycol | glycol | glycol | | sulfoxide |

TABLE 2
Ingredients of Strontium-doped Root Canal Filling Paste of Examples 12-16

| | Example No. | | | | |
|---|---|---|---|---|---|
| Component | Example 12 | Example 13 | Example 14 | Example 15 | Example 16 |
| (a) | 40% | 20% | 25% | 30% | 20% |
| | tricalcium | tricalcium | dicalcium | tricalcium | tricalcium |
| | silicate | silicate | silicate | silicate | silicate |
| (b) | 0.5% | 5% | 7% | 4% | 3% |
| | strontium | strontium | strontium | strontium | strontium |
| | phosphate | acetate | phosphate | chloride | nitrate |
| (c) | 30% | 40% | 35% | 37% | 33% |
| | columbium | bismuth | columbium | barium | iodoform |
| | monoxide | trioxide | dioxide | sulfate | |
| (d) | 9.5% | 10% | 5% calcium | 7% | 4% calcium |
| | tetracalcium | α-tricalcium | dihydrogen | β-tricalcium | dihydrogen |
| | phosphate | phosphate | phosphate | phosphate | phosphate |
| (e) | 20% | 25% | 28% | 22% | 40% |
| | polyethylene | polyethylene | polyethylene | polyethylene | polyethylene |
| | glycol | glycol | glycol | glycol | glycol |

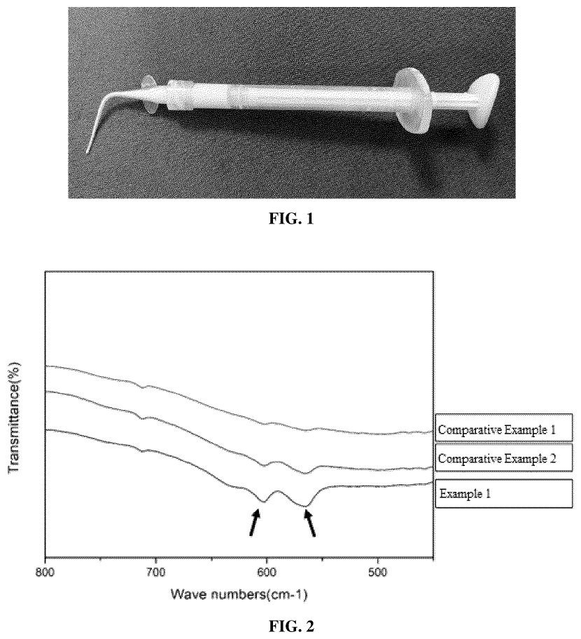

Examples 1-16 further respectively provide a preparation method for a strontium-doped root canal filling paste. The preparation methods of Examples 1-16 are the same, and the difference merely lies in changing ratios of relevant raw materials. Illustration is given by taking Example 1 as an example. Corresponding raw materials were accurately weighed with reference to the ratios provided in Table 1 and Table 2 above, and powders of the component (a) and the component (b) were put into a ball mill and ball-milled uniformly (a media-to-material ratio was 10:1, and a rotational speed was 350 r/min), so as to obtain a strontium-physically-doped calcium silicate mixed powder (strontium calcium silicate for short). Then the strontium calcium silicate, the component (c), the component (d) and the component (e) were put into a mixer and stirred uniformly, so as to obtain a uniformly mixed paste. Finally the mixed paste was dispensed into a medical syringe fitted with an injection hose needle, so as to obtain a syringe containing the strontium-doped root canal filling paste (see FIG. 1). 16groups of root canal filling paste were prepared in total in Examples 1˜16 by the same method.

### Comparative Examples 1-5

Comparative Examples 1-5 respectively provide a root canal filling paste, of which ingredients are shown in following Table 3.

TABLE 3
Ingredients of Root Canal Filling Paste

| | Example No. | | | | |
|---|---|---|---|---|---|
| | Comparative | Comparative | Comparative | Comparative | Comparative |
| Component | Example 1 | Example 2 | Example 3 | Example 4 | Example 5 |
| (a) | 37% | 37% strontium- | 22% | 42% | 10% |
| | tricalcium | doped tricalcium | tricalcium | tricalcium | tricalcium |
| | silicate | silicate | silicate | silicate | silicate |
| | | (Sr_{0.18}Ca_{2.82}SiO₅) | | | |
| (b) | 0 | 0 | 15% strontium | 0 | 0 |
| | | | phosphate | | |
| (c) | 33% | 33% | 33% | 33% | 60% |
| | zirconium | zirconium | zirconium | zirconium | zirconium |
| | oxide | oxide | oxide | oxide | oxide |
| (d) | 5% calcium | 5% calcium | 5% calcium | 0 | 5% calcium |
| | dihydrogen | dihydrogen | dihydrogen | | dihydrogen |
| | phosphate | phosphate | phosphate | | phosphate |
| (e) | 25% | 25% | 25% | 25% | 25% |
| | polyethylene | polyethylene | polyethylene | polyethylene | polyethylene |
| | glycol | glycol | glycol | glycol | glycol |

With regard to the preparation method for the root canal filling paste of Comparative Examples 1-11, if strontium compounds were not doped, the root canal filling paste was prepared through following steps: putting the component (a), the component (c), the component (d) and the component (e) in a mixer and stirring them uniformly, so as to obtain a uniformly mixed paste. Finally the mixed paste was dispensed into a medical syringe fitted with an injection hose needle, so as to obtain a syringe containing the root canal filling paste. If strontium compounds were doped, the root canal filling paste was prepared with reference to the preparation method in Example 1, and only types and ratios of the raw materials were changed correspondingly.

### Comparative Examples 6-11

Comparative Example 6-11 respectively provide a strontium-doped root canal filling paste, of which ingredients are shown in following Table 4.

TABLE 4
Ingredients of Strontium-doped Root Canal Filling Paste

| | Example No. | | | | | |
|---|---|---|---|---|---|---|
| | Comparative | Comparative | Comparative | Comparative | Comparative | Comparative |
| Component | Example 6 | Example 7 | Example 8 | Example 9 | Example 10 | Example 11 |
| (a) | 35% | 35% | 35% | 35% | 35% | 35% |
| | tricalcium | tricalcium | tricalcium | tricalcium | tricalcium | tricalcium |
| | silicate | silicate | silicate | silicate | silicate | silicate |
| (b) | 2% strontium | 2% strontium | 2% strontium | 2% strontium | 2% strontium | 2% strontium |
| | glass powder | sulfate | zirconate | aluminate | tungstate | molybdate |
| (c) | 33% | 33% | 33% | 33% | 33% | 33% |
| | zirconium | zirconium | zirconium | zirconium | zirconium | zirconium |
| | oxide | oxide | oxide | oxide | oxide | oxide |
| (d) | 5% calcium | 5% calcium | 5% calcium | 5% calcium | 5% calcium | 5% calcium |
| | dihydrogen | dihydrogen | dihydrogen | dihydrogen | dihydrogen | dihydrogen |
| | phosphate | phosphate | phosphate | phosphate | phosphate | phosphate |
| (e) | 25% | 25% | 25% | 25% | 25% | 25% |
| | polyethylene | polyethylene | polyethylene | polyethylene | polyethylene | polyethylene |
| | glycol | glycol | glycol | glycol | glycol | glycol |

### Detection Example 1

According to Pharmaceutical Industry Standard of the People's Republic of China: Dental Root Canal Sealing Materials (YY0717-2009), the properties of the root canal filling pastes prepared in Examples 1˜11, Comparative Examples 1-5 and Comparative Examples 6-11 were respectively detected, wherein detection items included appearance, fluidity, and curing time. Test results of the properties are shown in Table 5, Table 6 and Table 7.

TABLE 5
Detection Results (1)

| | Example No. | | | | | | | | | | | |
|---|---|---|---|---|---|---|---|---|---|---|---|---|
| Property | Ex. 1 | | Ex. 2 | | Ex. 3 | | Ex. 4 | | Ex. 5 | | Ex. 6 | |
| Appearance | Uniform | | Uniform | | Uniform | | Uniform | | Uniform | | Uniform | |
| | appearance, | | appearance, | | appearance, | | appearance, | | appearance, | | appearance, | |
| | without | | without | | without | | without | | without | | without | |
| | visible | | visible | | visible | | visible | | visible | | visible | |
| | foreign | | foreign | | foreign | | foreign | | foreign | | foreign | |
| | matters | | matters | | matters | | matters | | matters | | matters | |
| Fluidity | 26 | mm | 24 | mm | 24 | mm | 25 | mm | 24 | mm | 25 | mm |
| Curing Time | 2.5 | h | 3 | h | 3 | h | 3 | h | 3.5 | h | 3.5 | h |

| | Example No. | | | | | | | | | |
|---|---|---|---|---|---|---|---|---|---|---|
| Property | Ex. 7 | | Ex. 8 | | Ex. 9 | | Ex. 10 | | Ex. 11 | |
| Appearance | Uniform | | Uniform | | Uniform | | Uniform | | Uniform | |
| | appearance, | | appearance, | | appearance, | | appearance, | | appearance, | |
| | without | | without | | without | | without | | without | |
| | visible | | visible | | visible | | visible | | visible | |
| | foreign | | foreign | | foreign | | foreign | | foreign | |
| | matters | | matters | | matters | | matters | | matters | |
| Fluidity | 25 | mm | 24 | mm | 25 | mm | 24 | mm | 24 | mm |
| Curing Time | 3.5 | h | 3 | h | 3 | h | 3.5 | h | 3.5 | h |

TABLE 6
Detection Results (2)

| | Example No. | | | | | | | | | |
|---|---|---|---|---|---|---|---|---|---|---|
| | Comparative | | Comparative | | Comparative | | Comparative | | Comparative | |
| Property | Example 1 | | Example 2 | | Example 3 | | Example 4 | | Example 5 | |
| Appearance | Uniform appear- | | Uniform appear- | | Uniform appear- | | Uniform appear- | | Uniform appear- | |
| | ance, without | | ance, without | | ance, without | | ance, without | | ance, without | |
| | visible foreign | | visible foreign | | visible foreign | | visible foreign | | visible foreign | |
| | matters | | matters | | matters | | matters | | matters | |
| Fluidity | 25 | mm | 25 | mm | 26 | mm | 28 | mm | 18 | mm |
| Curing | 19 | h | 4 | h | 8 | h | 32 | h | 35 | h |
| Time | | | | | | | | | | |

TABLE 7
Detection Results (3)

| | Example No. | | | | | | | | | | | |
|---|---|---|---|---|---|---|---|---|---|---|---|---|
| | Comparative | | Comparative | | Comparative | | Comparative | | Comparative | | Comparative | |
| Property | Example 6 | | Example 7 | | Example 8 | | Example 9 | | Example 10 | | Example 11 | |
| Appearance | Uniform | | Uniform | | Uniform | | Uniform | | Uniform | | Uniform | |
| | appearance, | | appearance, | | appearance, | | appearance, | | appearance, | | appearance, | |
| | without | | without | | without | | without | | without | | without | |
| | visible | | visible | | visible | | visible | | visible | | visible | |
| | foreign | | foreign | | foreign | | foreign | | foreign | | foreign | |
| | matters | | matters | | matters | | matters | | matters | | matters | |
| Fluidity | 20 | mm | 22 | mm | 19 | mm | 21 | mm | 26 | mm | 24 | mm |
| Curing Time | 16 | h | 14 | h | 15 | h | 14 | h | 17 | h | 13 | h |

It can be seen from Table 5 and Table 6 that the strontium-doped root canal filling pastes provided in the examples of the present disclosure have a uniform appearance, without visible foreign matters. The fluidities were 24-26 mm, with no significant difference.

By comparing Examples 1 and 2 and Comparative Example 1 and Comparative Example 3, it can be seen that when a strontium doping amount is 0 (namely, without strontium doping), the curing time is 19 h. When 2% or 10% strontium phosphate is doped, the curing time can be greatly shortened. In the above, when the doping amount is 2%, the curing time is the shortest, only 2.5 h. However, when the doping amount is 15%, the curing time is increased to 8 h. It indicates that the strontium doping can significantly shorten the curing time, and it is not the case that a higher doping amount is better, while a high strontium doping amount on the contrary may prolong the curing.

It can be seen by comparing Example 1 and Comparative Example 2 that the physical doping of the strontium compound (strontium phosphate)+tricalcium silicate, compared with chemically doping a single component strontium-doped tricalcium silicate, greatly reduces the curing time of the prepared root canal filling paste.

It can be seen by comparing Comparative Example 1, Comparative Example 4 and Comparative Example 5 that when the content of the tricalcium silicate is reduced to 10% or increased to 42%, the properties are adversely affected.

It can be seen by comparing Example 1 and Examples 9˜11 that when a liquid phase is polyethylene glycol, propylene glycol, glycerol, dimethyl sulfoxide, etc., the root canal filling paste can be successfully prepared.

According to Table 7, it can be seen from the comparison between Example 1, Comparative Example 1 and Comparative Examples 6-11 that not all doping of strontium compounds can achieve very good effects. The improvement on the curing time is less for Comparative Examples 6-11.

### Detection Example 2

In vitro Test for Biological Activity of Strontium-doped Root Canal Filling Paste

Samples of the strontium-doped root canal filling pastes prepared in Example 1, Comparative Example 1 and Comparative Example 2 were compared and tested for remineralization property with reference to an infrared spectroscopy method in T/CSBM 0026-2022 In vitro mineralization test method of bioactive glass. Infrared characteristic absorption peaks of mineralization products of different reactions were analyzed, wherein judgment basis was whether a P-O bending vibration absorption peak representing a crystalline state appeared at 610 cm˜600 cm and 560 cm˜550 cm in a Fourier transform infrared spectrum, and intensities of mineralization absorption peaks were compared. Infrared spectroscopy test results of the root canal filling pastes after 4 h soaking in SBF are shown in FIG. 2.

According to FIG. 2, it can be seen that infrared curves of all samples have double vibration absorption peaks at 610 cm˜600 cm and 560 cm˜550 cm (indicated by arrows in FIG. 2), which indicates that all the root canal filling pastes prepared in Example 1, Comparative Example 1 and Comparative Example 2 have biological activity/mineralization characteristic. It can be seen from comparison of peak intensities that the peak intensity of Comparative Example 1 is very weak, indicating that the root canal filling paste without strontium doping has very low biological activity. The peak intensity of Comparative Example 2 is enhanced, indicating that the root canal filling paste prepared by using the strontium-doped tricalcium silicate formed by chemically doping has improved biological activity. The peak intensity of Example 1 is greatly improved, indicating that the root canal filling paste prepared by physically mixing and doping the strontium compound and tricalcium silicate has the best biological activity. This may be due to the way of strontium doping by physical mixing, where strontium elements can be quickly released, thereby realizing true induced remineralization.

### Detection Example 3

Detection of the bonding strength of the strontium-doped root canal filling paste with the root canal wall

30 extracted teeth (premaxillary teeth, and single root canal) were collected. The extracted teeth were subjected to root canal preparation through a standard root canal preparation procedure, and were respectively filled with the root canal filling pastes prepared in Example 1, Comparative Example 1 and Comparative Example 2 in cooperation with gutta percha point by single-cone filling.

All the samples were embedded in wax blocks, and cut at intervals of 1 mm in a direction from root to crown by a low-speed slice machine, with a blade being perpendicular to a long axis of teeth. 9 teeth sheets with a thickness of (1.00±0.20) mm were obtained from each sample, and a fifth sheet was taken as an experimental sample. Thickness h (accurate to 0.01 mm) of each tooth sheet was measured by a vernier caliper, a crown diameter D1 and a root diameter D2 of each tooth sheet were measured under a microscope for all the sheets, and an adhesive area A was calculated according to a formula:

$$A = 0.5 \times π \times ( D 1 + D 2 ) \times h$$

Each sample tooth sheet was fixed on a fixture dedicated to push-out test by sticky wax, and then was placed on a loading platform of a mechanical testing machine. A matched loading head was chosen. The loading head was adjusted in position so that it is perpendicular to the tooth sheet and only contacts the filling material. A displacement speed of crossbeam was controlled at 0.05 mm/min. Load F when the filling material moved was recorded. Adhesive strength was calculated according to formula P=F/A. Mean values of adhesion strength results are shown in Table 8.

TABLE 8
Detection Results

| Group | Bonding Strength |
|---|---|
| Example 1 | 4.06 MPa |
| Comparative | 1.98 MPa |
| Example 1 | |
| Comparative | 2.85 MPa |
| Example 2 | |

It can be seen from Table 8 that Example 1 and Comparative Example 2 both render higher bonding strength to the root canal wall than Comparative Example 1, which may be due to improved mineralization property of the samples by the strontium doping. It can be seen from comparison between Example 1 and Comparative Example 2 that the bonding strength with the root canal wall in Example 1 is significantly higher than that of Comparative Example 2, which indicates that the root canal filling paste prepared by mixed doping of strontium compound and tricalcium silicate is best bonded with the root canal wall. This may be due to the best mineralizability/biological activity of the root canal filling paste prepared by the strontium doping in a physical mixing manner, and the hydroxyapatite formed by the mineralization penetrates into dentine pores of the root canal wall, so that the bonding strength is higher, and the root canal sealing ability is also better.

The above are merely preferred examples of the present disclosure, but are not intended to limit the present disclosure. For those skilled in the art, various modifications and changes could be made to the present disclosure. Any amendments, equivalent replacements, improvements and so on, made within the spirit and principle of the present disclosure, should be covered within the scope of protection of the present disclosure.

What is claimed is:

1. A strontium-doped root canal filling paste, comprising a component (a) and a component (b), wherein component (a) is selected from calcium silicate compounds, the component (b) is selected from strontium compounds, and wherein component (a) accounts for 20%-40% by weight of the root canal filling paste, component (b) accounts for 0.5%-10% by weight of the root canal filling paste; and component (a) and component (b) are doped in a physical doping manner.

2. The strontium-doped root canal filling paste according to claim 1, wherein component (a) accounts for 25%-35% by weight of the root canal filling paste.

3. The strontium-doped root canal filling paste according to claim 2, wherein component (b) accounts for 2-5% by weight of the root canal filling paste.

4. The strontium-doped root canal filling paste according to claim 1, wherein the calcium silicate compounds are any one or a combination of any two or more selected from the group consisting of tricalcium silicate, dicalcium silicate and monocalcium silicate.

5. The strontium-doped root canal filling paste according to claim 4, wherein the strontium compound is any one or a combination of any two or more selected from the group consisting of strontium chloride, strontium phosphate, strontium nitrate, strontium fluoride, strontium acetate and strontium ranelate.

6. The strontium-doped root canal filling paste according to claim 1, further comprising component (c), and component (c) is a radiation inhibitor.

7. The strontium-doped root canal filling paste according to claim 6, wherein the radiation inhibitor is selected from the group consisting of metal oxides, metal sulfate compounds and polyhalogenated C1-C3 alkyls.

8. The strontium-doped root canal filling paste according to claim 7, wherein the radiation inhibitor is selected from the group consisting of transition metal oxides, metal sulfate compounds and polyiodine-substituted C1-C3-alkyls.

9. The strontium-doped root canal filling paste according to claim 8, wherein the radiation inhibitor is any one or a combination of any two or more selected from the group consisting of niobium oxides, zirconium oxides, bismuth oxides, tantalum oxides, barium sulfate and iodoform.

10. The strontium-doped root canal filling paste according to claim 6, wherein component (c) accounts for 30%-40% by weight of the root canal filling paste.

11. The strontium-doped root canal filling paste according to claim 1, further comprising a component (d), and the component (d) is a calcium phosphate compound.

12. The strontium-doped root canal filling paste according to claim 11, wherein the calcium phosphate compound is any one or a combination of any two or more selected from the group consisting of α-tricalcium phosphate, β-tricalcium phosphate, tetracalcium phosphate, calcium dihydrogen phosphate and hydroxyapatite.

13. The strontium-doped root canal filling paste according to claim 11, wherein component (d) accounts for 2%-10% by weight of the root canal filling paste.

14. The strontium-doped root canal filling paste according to claim 1, further comprising a component (e), and the component (e) is an organic solvent which can be mixed with the component (a) and the component (b) to form a paste.

15. The strontium-doped root canal filling paste according to claim 14, wherein the organic solvent is any one or a combination of any two or more selected from the group consisting of alcohol solvents, polymer solvents and sulfoxide solvents.

16. The strontium-doped root canal filling paste according to claim 15, wherein the organic solvent is selected from the group consisting of C2-C5 polyols, poly C2-C5 polyols, polypyrrolidine compounds and C2-C5 sulfoxide solvents.

17. The strontium-doped root canal filling paste according to claim 14, wherein component (e) accounts for 20%-40% by weight of the root canal filling paste.

18. A method of treating or preventing dental pulp disease, comprising applying the strontium-doped root canal filling paste according to claim 1 to the dental pulp region of a tooth root or root canal region of a mammal requiring such treatment.

19. A preparation method for the strontium-doped root canal filling paste of claim 1, comprising: physically mixing component (a) and component (b) uniformly.

20. The preparation method according to claim 19, comprising: ball-milling and mixing component (a) and component (b) to form a mixed powder; and then mixing the mixed powder with at least one of component (c), a component (d) and a component (e) uniformly.
